(19) United States
(12) Patent Application Publication
(10) Pub. No.: US 2017/0234798 A1
(43) Pub. Date: Aug. 17, 2017

(54) METHOD AND DEVICE FOR DIAGNOSING VIRAL INFECTION USING TEARDROP

(71) Applicant: UNIVERSITY-INDUSTRY COOPERATION GROUP OF KYUNG HEE UNIVERSITY, Gyeonggi-do (KR)

(72) Inventors: Sam Jin CHOI, Seoul (KR); Jae Ho SHIN, Seoul (KR); Hun Kuk PARK, Seoul (KR); Kyung Hyun JIN, Seoul (KR)

(21) Appl. No.: 15/329,101

(22) PCT Filed: May 12, 2015
(86) PCT No.: PCT/KR2015/004736

§ 371 Date: Jan. 25, 2017

(30) Foreign Application Priority Data
Jul. 29, 2014 (KR) 10-2014-0096695

(51) Int. Cl. G01N 21/65; A61B 5/00; G01N 33/487

(52) U.S. Cl. CPC G01N 21/658; G01N 33/487; A61B 5/0075; G01N 2201/12

(57) ABSTRACT

The present invention relates to a method for providing information on the presence of viral infection, comprising: a first step of preparing a dried tear sample on a substrate; a second step of measuring a Raman spectrum from the dried tear sample; a third step of extracting Gaussian sub-peaks by deconvolution of the measured Raman spectrum; a fourth step of deriving a log value for the relative intensity ratio of a peak corresponding to an amide III β-sheet and a peak corresponding to C—H deformation; and a fifth step of determining the sample as normal if the derived value is positive and as infected if the derived value is negative.

### TECHNICAL FIELD

The present invention relates to a method for providing information on the presence of viral infection by measuring the Raman spectrum of a dried tear sample prepared on a substrate and by extracting multiple Gaussian peaks therefrom to evaluate the intensity ratio of two specific wavelengths; and to a diagnostic device for viral infection using the same.

### BACKGROUND ART

Currently, for the diagnosis of infectious diseases, methods comprising multiple steps of collecting and culturing cells, collecting genes therefrom, and amplifying the genes by polymerase chain reaction (PCR) to confirm, are used. While these methods require much time and effort, it is important for such infectious diseases to be quickly diagnosed and properly treated because they are contagious in many cases. Therefore, a method for quickly and easily diagnosing infectious disease is required.

Tear analysis methods based on Raman spectroscopy have recently been studied for the research on infectious ocular surface diseases. For example, Korean Patent No. 10-1336478 discloses detection of viral particles in tear films using surface-enhanced Raman spectroscopy (SERS).

However, in the case of the tear analysis methods based on the Raman spectroscopy of prior art, they diagnose the presence or absence of a virus in a sample by analyzing the difference of overall SERS spectrum patterns, it is difficult to analyze the difference because they compare the entire spectrum patterns, and there is a problem in that the boundary for accurately diagnosing an infection is not clear.

### DISCLOSURE

### Technical Problem

It is an object of the present invention to provide a method and device for diagnosing viral infections, which are derived from the technical background described above, and which can diagnose infectious diseases quickly and simply.

Another object of the present invention is to provide a stand-alone diagnostic device for viral infection, which can be used for the diagnosis of infectious diseases at clinical sites; and a method for diagnosing viral infection using the same.

Another object of the present invention is to provide a method and device for diagnosing viral infection, in which the presence of viral infection can be accurately diagnosed by tear analysis methods based on Raman spectroscopy.

### Technical Solution

The present invention provides a method for providing information on the presence of viral infection, comprising: a first step of preparing a dried tear sample on a substrate; a second step of measuring a Raman spectrum from the dried tear sample; a third step of extracting Gaussian sub-peaks by deconvolution of the measured Raman spectrum; a fourth step of deriving a log value for the relative intensity ratio of a peak corresponding to an amide III β-sheet and a peak corresponding to C—H deformation by Equation 1 below; and a fifth step of determining the sample as normal if the derived value is positive and as infected if the derived value is negative:

$$AC = {log}_{10} ( \frac{{I}_{amide III} {β}_{sheet}}{{I}_{C \text{-} Hdeform}} ) . \qquad [ Equation 1 ]$$

Further, the present invention provides a diagnostic device for viral infection, comprising: a detection substrate capable of providing a dried tear sample by applying a teardrop thereon; a signal measuring unit for measuring a Raman signal from the inserted detection substrate; a peak deconvolution unit for separating the measured Raman peaks into Gaussian sub-peaks; a data processing unit for deriving a log value for a relative ratio of two peaks appearing at specific wavelengths among the separated Gaussian sub-peaks; and a display unit for showing the derived value.

### Advantageous Effects

According to the present invention, about 10 overlapped peaks appearing in a range of 1200 cm⁻¹ to 1500 cm⁻¹ are separated into single Gaussian peaks from the spectrum obtained using drop-coating deposition surface-enhanced Raman spectroscopy (DCD-SERS) in which surface-enhanced Raman scattering and drop-coating deposition are fused, and the relative intensity ratio of two specific peaks therefrom, particularly, peaks appearing at 1342 cm⁻¹ and 1242 cm⁻¹, can be evaluated to confirm the presence of adenoviral infection.

The method for diagnosing viral infection of the present invention can diagnose viral infection faster than conventional PCR methods, and the present invention can provide a stand-alone diagnostic device which can be used for the diagnosis of infectious diseases at clinical sites.

### BRIEF DESCRIPTION OF THE DRAWINGS

FIGS. 1A-D show surface conditions of the two types of substrates used in an exemplary embodiment of the present invention.

FIGS. 2A-B show the SERS activity of the two types of the substrates observed using a balanced salt solution (BSS).

FIGS. 3A-B show representative DCD-SERS spectra of tear samples collected from non-infected persons and adenoviral conjunctivitis-confirmed patients.

FIG. 4 shows DCD-SERS spectra measured using BSS as a negative control.

FIGS. 5A-D show light microscope (LM) photographs of each zone of dried tear samples compartmented to obtain reliable DCD-SERS spectra in an exemplary embodiment of the present invention.

FIGS. 6A-F show results analyzing the characteristics of DCD-SERS spectra depending on the amount of tears used from each zone of FIGS. 5A-D.

FIGS. 7A-D show results of interpreting the movement of particles from a center to a ring part with respect to an arbitrary time change using a finite element analysis.

FIGS. 8A-B shows superimposed DCD-SERS spectra measured at 10 different points in the same zone of the same sample.

FIG. 9 shows DCD-SERS spectra and characteristic Raman peaks of the samples obtained from non-infected persons and adenoviral conjunctivitis patients.

FIGS. 10A-D show loading plots of three PC profiles for the non-infected group and the adenoviral conjunctivitis patient group in a central (C) zone.

FIGS. 11A-D shows DCD-SERS spectra measured at wavelengths in a range of 1200 cm⁻¹ to 1500 cm⁻¹ for the C zone and a primary ring (R) zone and 10 Gaussian sub-peaks extracted therefrom. (A) and (C), and (B) and (D) show results for the samples taken from the non-infected persons and infected patients, respectively.

FIG. 12 schematically shows an entire system for diagnosing viral infection using a portable diagnostic device for viral infection and a method for providing information on the presence of viral infection according to the present invention.

### BEST MODE

The present invention provides a method for providing information on the presence of viral infection, comprising: a first step of preparing a dried tear sample on a substrate; a second step of measuring a Raman spectrum from the dried tear sample; a third step of extracting Gaussian sub-peaks by deconvolution of the measured Raman spectrum; a fourth step of deriving a log value for the relative intensity ratio of a peak corresponding to an amide III β-sheet and a peak corresponding to C—H deformation by Equation 1 below; and a fifth step of determining the sample as normal if the derived value is positive and as infected if the derived value is negative:

$$AC = {log}_{10} ( \frac{{I}_{amide III} {β}_{sheet}}{{I}_{C \text{-} Hdeform}} ) . \qquad [ Equation 1 ]$$

The present invention is based on the first finding that viral infection can be diagnosed by evaluating the peak intensity ratio at two specific wavelengths by deconvoluting the Raman spectrum for dried tear samples, in which about 10 Gaussian peaks appear by being overlapped, into individual Gaussian peaks. For example, in the case of patients suffering from conjunctivitis due to adenoviral infection, by confirming that a log value of the relative ratio of the peak intensity at 1242 cm⁻¹ to the peak intensity at 1342 cm⁻¹ changed from a positive value to a negative value, these two peaks were identified as useful parameters for the diagnosis of adenoviral infection, and a method for diagnosing infection using the same was suggested.

Preferably, the first step may be performed by drop-coating deposition (DCD).

Preferably, the second step may be performed by surface-enhanced Raman spectroscopy.

Preferably, the substrate may be a support coated with nanoparticles. By using a nanoparticle-coated support, the sensitivity of measurements can be improved by inducing surface-enhanced Raman scattering. In general, Raman scattering is excellent in selectivity, but has a disadvantage in that detection is not easy due to weak signal intensity as compared with other optical detection methods such as absorption, fluorescence, etc. Therefore, in order to overcome this, it is necessary to use a highly sensitive detector, or a method capable of increasing the signals is needed. Accordingly, by using a support coated with nanoparticles, Raman signals generated by the surface enhancement effect due to the nanoparticles can be enhanced, and thus measurements can be performed without the aid of a special detector.

Preferably, the measurements may be performed at the central (C) zone, middle (M) zone, or secondary ring (T) zone of the dried tear sample.

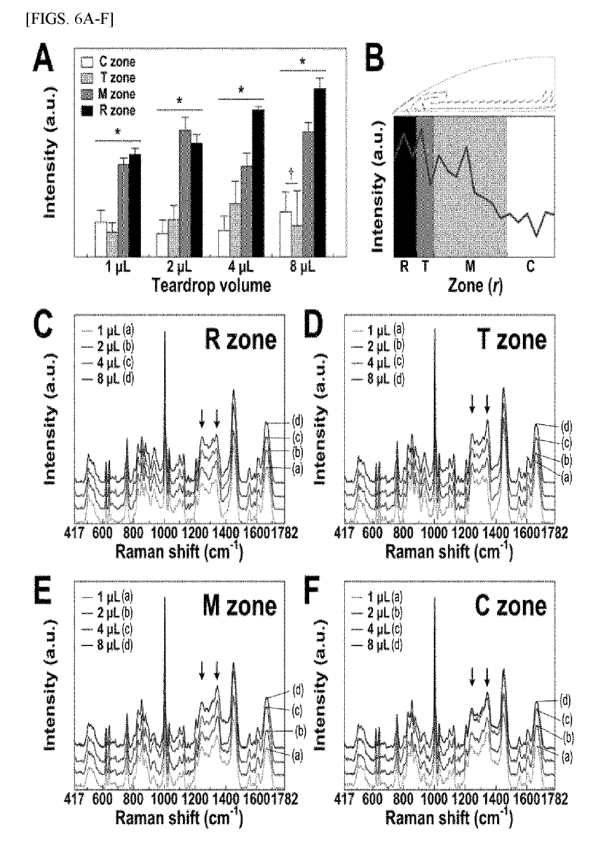

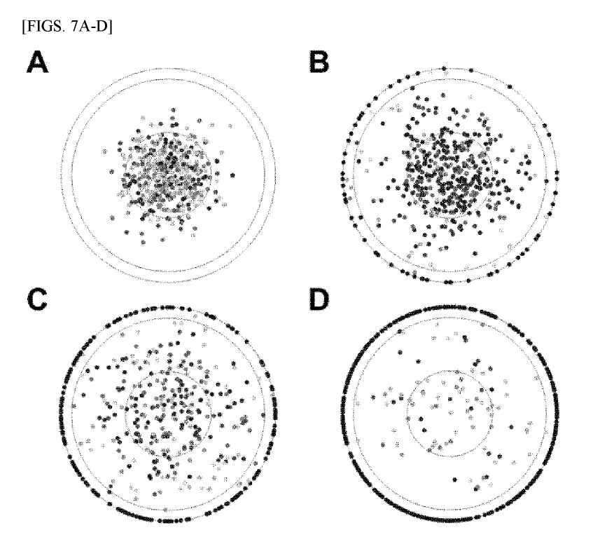

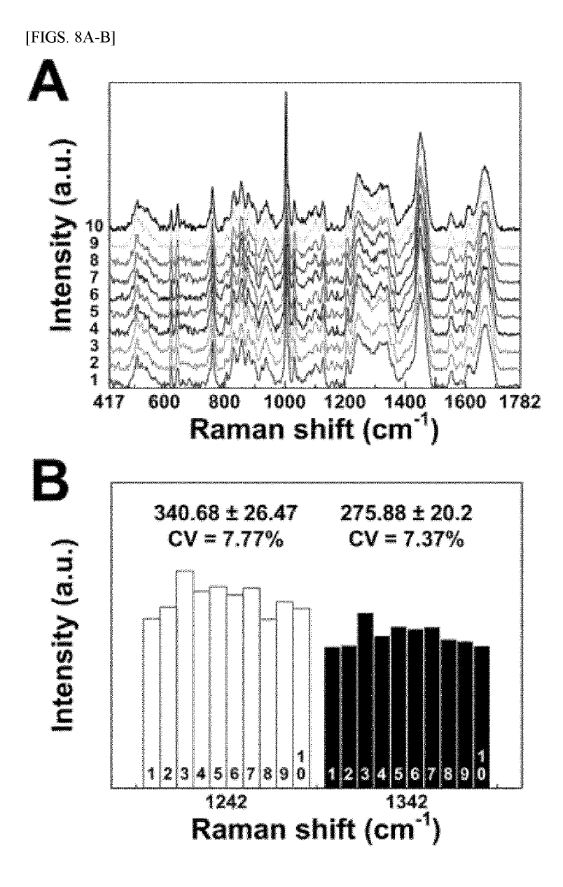

In a specific exemplary embodiment of the present invention, as a result of measuring and analyzing the Raman spectra at the four zones of the dried tear samples, namely, the C, M, T, and R zones, a significant change in relative signal intensity was observed at two selected wavelengths at C, M, and T zones, but the change observed at the R zone was negligible (FIGS. 6A-F). Therefore, a more sensitive and accurate diagnosis may be possible by measuring at the C, M, or T zone.

Preferably, the peak corresponding to the amide III β-sheet may appear in a range of 1242±10 cm⁻¹, and the peak corresponding to C—H deformation may appear in a range of 1342±10 cm⁻¹, respectively.

Preferably, the method for providing information of the present invention may provide information on the presence of adenoviral infection.

In a specific exemplary embodiment of the present invention, tear samples from adenoviral conjunctivitis-confirmed patients and from non-infected persons were compared, and as a result, it was confirmed that in the Raman spectrum of the non-infected samples, the log value of the intensity ratio of the peak at 1242 cm⁻¹ corresponding to the amide III β-sheet to the peak at 1342 cm⁻¹ corresponding to C—H deformation was always positive, but in the spectrum of adenoviral conjunctivitis-confirmed patients, the ratio was remarkably decreased, showing a negative log value. That is, a spectrum appearing by about 10 overlapped peaks in the range of 1200 cm⁻¹ to 1500 cm⁻¹ were resolved into single Gaussian peaks, and by evaluating the relative intensity ratio of two specific peaks among those peaks, in particular, the peaks appearing at 1342 cm⁻¹ and 1242 cm⁻¹, it was confirmed that it was possible to determine the presence of adenoviral infection.

Further, the present invention provides a diagnostic device for viral infection, comprising: a detection substrate capable of providing a dried tear sample by applying a teardrop thereon; a signal measuring unit for measuring a Raman signal from the inserted detection substrate; a peak deconvolution unit for separating the measured Raman peaks into Gaussian sub-peaks; a data processing unit for deriving a log value for a relative ratio of two peaks appearing at specific wavelengths among the separated Gaussian sub-peaks; and a display unit for showing the derived value.

Preferably, the diagnostic device of the present invention may further comprise an input unit into which the detection substrate is inserted.

Preferably, in the diagnostic device of the present invention, the signal measuring unit may comprise a light source and photon detector, and optionally further comprise a mirror, lens, and a filter.

### DETAILED DESCRIPTION OF THE EMBODIMENTS

Hereinbelow, the constitution and effects of the present invention will be described in detail with accompanying exemplary embodiments. However, the exemplary embodiments disclosed herein are only for illustrative purposes and should not be construed as limiting the scope of the present invention.

### Example 1: Sample Collection and Measurement

Among patients who visited Kyung Hee University Hospital, tear samples were collected from 8 patients (36±14 yr) who had been confirmed with adenoviral conjunctivitis and 8 normal persons (33±8 yr) with their consent. The present study has passed the IRB KMCIRN1401-02 at Kyung Hee University.

Tear collection was performed for 5 minutes at a nasoinferior conjunctival sac using a polyester-fiber rod (Transorb Wick, USA) with a diameter of 4 mm and a length of 10 mm without external stimulation. The rod which was removed from the eye was placed in an Eppendorf tube and centrifuged at 8,000 rpm for 15 minutes to remove the rod, and thereafter, it was sealed with parafilm (Pechiney, Plastic Packing Company, USA) and stored at −70° C. for 24 hours. It did not exceed 24 hours until the measurement was performed according to the present invention.

In order to obtain Raman spectra from the collected tear samples, a DCD-SERS spectral method was used, in which surface-enhanced Raman scattering (SERS) and drop-coating deposition (DCD) are fused. Specifically, a 50 nm Au-coated anodized aluminum oxide (AAO) nanodot array substrate and a commercially available 2.5 nm Ti- and 50 nm Au-coated Au.0500.ALSI (Platypus Technologies, USA) substrate were used. Approximately 2 μL of tear was dropped on a clean substrate and dried to prepare samples for measurement. A SENTERRA confocal Raman system (Bruker Optics Inc., USA) equipped with a 785 nm diode laser source with a 200 mW output was used. In addition, it was possible to measure with a portable Raman. The examination was performed for 30 seconds by laser-irradiating the dried tear, which was sectioned into four zones (C, M, T, and R zones, respectively, from the center), according to known methods. The measured spectra were in a range of 417 cm⁻¹ to 1782 cm⁻¹, and the central spectrum was 1200 cm⁻¹.

### Example 2: Diagnosis

2.1. Diagnostic Marker 1: AC

As shown in the Equation below, the log value of the ratio of the Raman intensity at a wavelength of 1242 cm⁻¹ corresponding to an amide III β-sheet relative to the Raman intensity at a wavelength of 1342 cm⁻¹ corresponding to C—H deformation was defined as an AC biomarker (see the Equation below). Whereas in non-infected normal tear, the amide III β-sheet at a wavelength of 1242 cm⁻¹ always had a greater value than the C—H deformation at a wavelength of 1342 cm⁻¹ so that the AC diagnostic marker always showed a positive value, in the case of conjunctivitis patients infected with adenovirus, the relative intensity of the peak at 1342 cm⁻¹ was increased, and the AC marker showed a negative value.

$$AC = {log}_{10} ( \frac{{I}_{1242}}{{I}_{1342}} )$$

In the Equation above, I₁₂₄₂ and I₁₃₄₂ are Raman intensities at wavelengths of 1242 cm⁻¹ and 1342 cm⁻¹, respectively. The above calculation was performed using MATLAB software.

2.2 Diagnostic Marker 2: Principal Component Analysis (PCA) Algorithm

Principal component analysis is a data processing technique that is useful for visualization and feature extraction of data, as well as dimensional reduction of feature vectors for reducing high dimensional feature vectors to low dimensional feature vectors. Three DCD-SERS spectra, each defined at 1242 cm⁻¹, 1342 cm⁻¹, and 1448 cm⁻¹, were used as inputs for a transfer function to detect the presence of adenoviral infection. Specifically, three vectors [1242 cm⁻¹, 1342 cm⁻¹], [1242 cm⁻¹, 1448 cm⁻¹], and [1342 cm⁻¹, 1448 cm⁻¹], which were normalized by the Z-score method, were used as inputs for the proposed transfer function. The performance of the principal component analysis was evaluated by the receiver operating characteristic curve (ROC curve) analysis, and the algorithm therefor was implemented in MATLAB software.

2.3 Diagnostic Marker 3: Deconvolution Method for Multiple Gaussian Peaks (MGPs)

In order to distinguish the difference between the normal condition and conjunctivitis due to adenoviral infection, a method for resolving multiple Gaussian peaks from the DCD-SERS spectrum was used. That is, the discrete version of a single Gaussian function can be defined by the Equation below:

$${g}_{k} ( f ) = {H}_{k} exp ( \frac{{( f - {f}_{k} )}^{2}}{2 {w}_{k}^{2}} )$$

In the Equation above, Hₖ is the amplitude of the single Gaussian function, fₖ is a maximum frequency position of the single Gaussian function, and wk is a half-width of the single Gaussian function.

The Gaussian curve of the optimized spectrum by using the above Equation can be expressed as the sum of Gaussian functions as shown by the Equation below.

$$G ( f ) = {\sum }_{k = 1}^{m} {g}_{k} ( f )$$

In the Equation above, m is the total number of Gaussian functions.

The DCD-SERS spectrum in the range of 1200 cm⁻¹ to 1500 cm⁻¹ was used as the input for the multi-Gaussian model for feature peak extraction using the above equation. In order to extract multiple Gaussian peaks (MGPs) from the measured spectrum, m=10, that is, 10 Gaussian peaks were selected to have 30 cm⁻¹ wavelength intervals within the range. From the four zones of a dried teardrop, wavelength shift (Raman shift), amplitude (Raman intensity), half-width, and area of Gaussian peaks were extracted and evaluated. An algorithm for extracting multiple Gaussian feature peaks using Gaussian resolution was also implemented in MATLAB software.

In order to compare the differences in mean values between two groups, for statistical analysis with a basic expression of mean and standard deviation, two-tailed Student's t-test method was used, and the intensity of the morphological DCD-SERS spectrum of the dried teardrop was analyzed using one-way analysis of variance (ANOVA). The Student-Newman-Keuls test was used for post hoc comparison. In order to evaluate the analytical efficiency of the AC biomarker, clinical analyses such as sensitivity, specificity, accuracy, prevalence, and error rates were used, and in order to evaluate the efficiency of the principal component analysis biomarker and the optimal limit of each variable, an ROC analysis method such as AUC (bottom area of ROC curve) was used. P values less than 0.05 were considered statistically significant.

<Result>

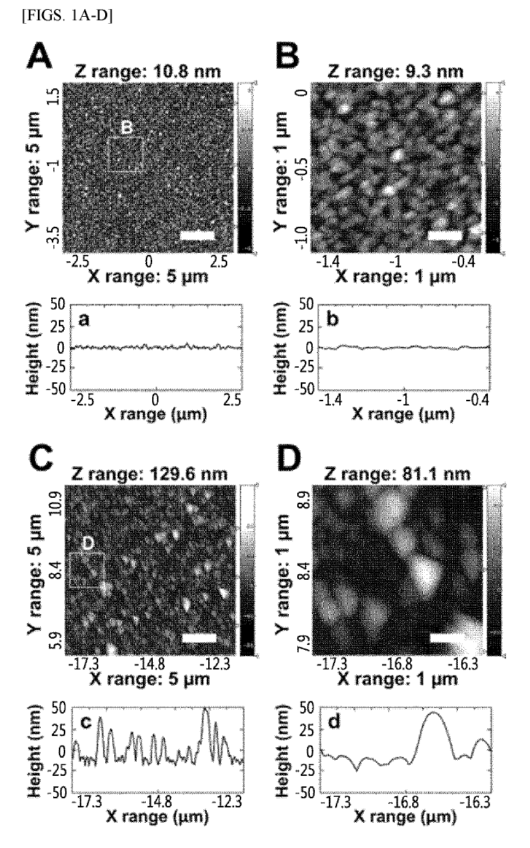

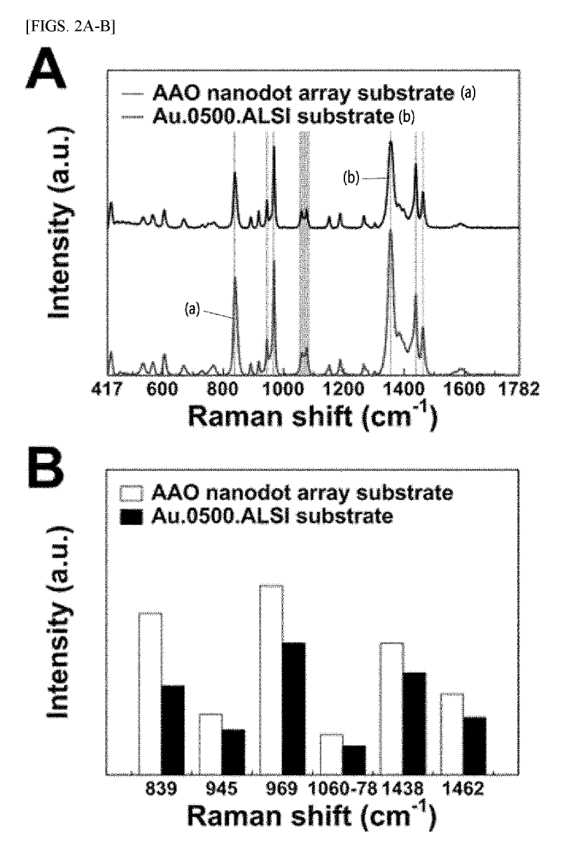

First, the surface characteristics of the two substrates used in the present invention, namely, a 50 nm Au-coated anodized aluminum oxide nanodot array substrate and a 2.5 nm Ti- and 50 nm Au-coated Au.0500.ALSI substrate, were observed using AFM, and the results are shown in FIGS. 1A-D.

For the surface characteristics analysis, NANOS N8 NEOS (Bruker, Germany), which is a tapping mode AFM device, was used, and as a result of analyzing the surface profile of the two types of the SERS substrates used, it was confirmed that the surface roughness characteristics of the 2.5 nm Ti- and 50 nm Au-coated Au.0500.ALSI substrate were reduced by 10 times compared to that of the 50 nm Au-coated anodized aluminum oxide nano-dot array substrate.

TABLE 1

| Surface profile | | | | |
|---|---|---|---|---|
| parameter | Au.0500.ALSI | | AAO nanodot array | |
| Coating | 2.5 nm Ti & 50 nm Au | | 50 nm Au | |
| Substrate | Aluminosilicate | | AAO-based | |
| | | | nanodot array | |
| Surface roughness | 5 μm × | 1 μm × | 5 μm × | 1 μm × |
| parameter | 5 μm | 1 μm | 5 μm | 1 μm |
| Mean roughness | 1.0 ± 0.3 | 0.9 ± 0.2 | 10.8 ± 2.3 | 11.9 ± 0.9 |
| (nm) | | | | |
| RMS roughness | 1.2 ± 0.3 | 1.1 ± 0.2 | 14.4 ± 3.7 | 15.4 ± 1.2 |
| (nm) | | | | |
| Peak-to-peak | 10.8 ± 1.1 | 9.3 ± 0.8 | 129.6 ± 12.6 | 81.1 ± 7.5 |
| height roughness | | | | |
| (nm) | | | | |

*AAO, anodized atuminum oxide; RMS, root-mean-square.

SERS activity of the above-mentioned two types of the substrates was observed using a balanced salt solution (BSS) used for eye washing in clinical practice. As a result, as shown in FIGS. 2A-B, it is known that there are seven prominent Raman bands in the wavelength bands of 839 cm⁻¹ (symmetric C—C—C stretching vibration of a proline ring), 945 cm⁻¹ and 969 cm⁻¹ (symmetric C—C stretching vibration of an acetate anion), 1060 cm⁻¹ to 1078 cm⁻¹ (symmetric C—N stretching vibration), 1356 cm⁻¹ (symmetric bending vibration of a methyl (CH₃) group), and 1438 cm⁻¹ and 1462 cm⁻¹ (asymmetric deformation of a methyl (CH₃) group or symmetrical deformation of a methylene (CH₂) group) (Podstawka, E. et al., Biopolymers, 2006, 83: 193-203; Musumeci, A. W. et al., Spectrochim. Acta A Mol. Biomol. Spectrosc., 2007, 67: 649-661).

Although the two types of the SERS substrates exhibited a similar spectral pattern, two-fold stronger intensity was exhibited in an AAO nanodot array substrate. Overall, the AAO nanodot array substrate exhibited more excellent nanostructure and DCD-SERS activity than the commercially available Au.0500.ALSI substrate.

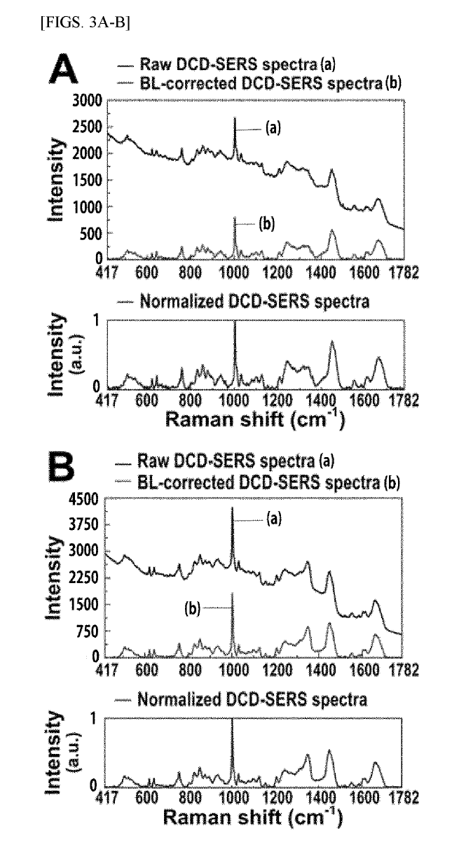

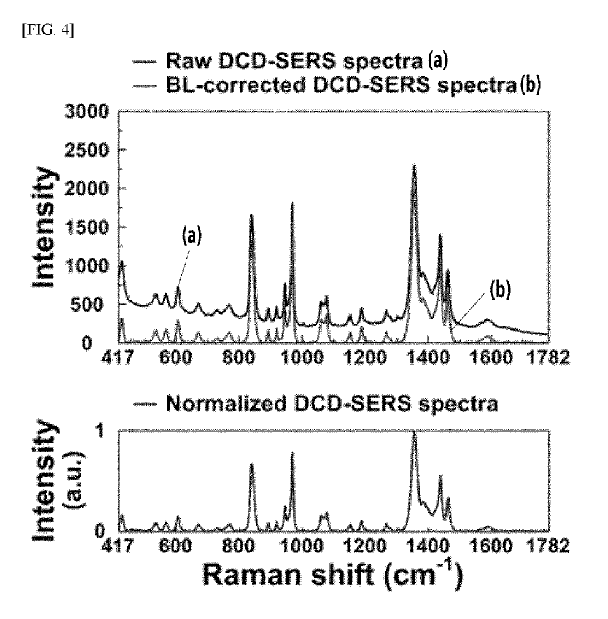

In order to reduce deviations between data by collecting data in various conditions, a pre-processing treatment was performed on the DCD-SERS spectrum. First, representative DCD-SERS spectra of tear samples collected from non-infected persons and adenoviral conjunctivitis-confirmed patients are shown in FIGS. 3A-B. As shown in FIGS. 3A-B, each DCD-SERS spectrum exhibited intrinsic vibration characteristics of tear samples. The DCD-SERS spectrum with the background signal subtracted (red) provided more definite Raman peak information than the spectrum containing the background signal (black). However, it was not possible to quantitatively compare signals from non-infected persons (FIG. 3A) and conjunctivitis patients (FIG. 3B) even in the DCD-SER spectrum, in which background signals were subtracted, by Raman intensity differences. However, qualitative and quantitative comparisons were only possible for the normalized DCD-SERS spectrum (blue).

As a negative control group, the DCD-SERS spectrum measured using BSS is shown in FIG. 4. It was confirmed from FIG. 4 that the DCD-SERS for BSS exhibited lower background signals than the spectra measured for the previous two samples. As in the experimental group, the background signal and normalized DCD-SERS spectrum provided clear Raman peak information, and from the top of the Figure, it was confirmed that qualitative or quantitative comparison of non-infected and adenoviral conjunctivitis samples was possible.

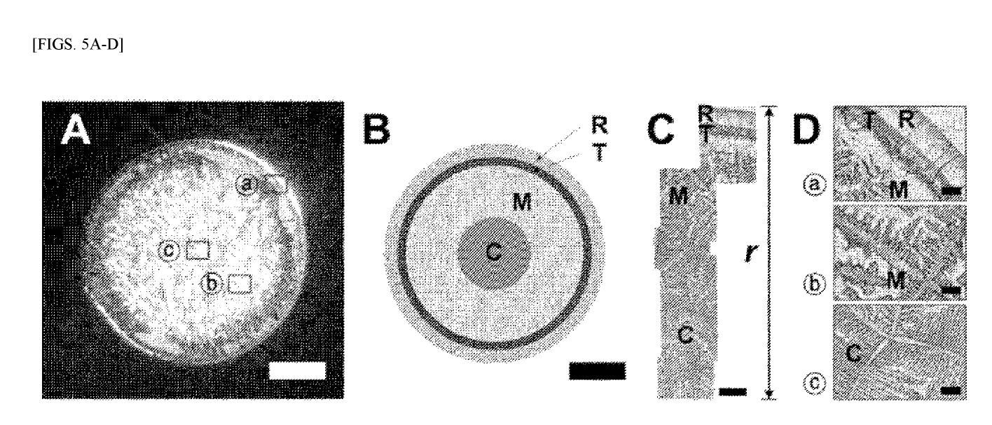

In all experiments, 2 μL of tear was used, and the total drying time was 20 minutes, from which dried tear samples having a diameter of approximately 4 mm were obtained. In order to obtain more reliable DCD-SERS spectra for hardware implementation, DCD-SERS spectra were measured and compared according to the different zones of dried tear. As shown in FIGS. 5A-D, the dried tear samples were divided into three zones, namely, R, M, and C zones, and the ring part located at the outermost region was further subdivided into R and T zones to be observed. FIGS. 5A-D show photographs of the respective zones taken by light microscope (LM).

Furthermore, the characteristics of the DCD-SERS spectrum depending on the amount of tear used from the respective zones were determined, and the results are shown in FIGS. 6A-F. In the case of using approximately 1 μL of tear drops, the DCD-SERS spectra in the C and T zones exhibited very low intensity, while the intensity in the R and M zones was strong. As a result of the ANOVA test (p<0.001, F-ratio=233.32) and post-verification test (SNK test; p<0.05), the DCD-SERS spectral intensity in the four zones showed significant differences. A similar pattern was also observed when using an increased amount (4 μL and 8 μL) of tear. That is, the signal intensity decreased linearly from the R zone to the C zone.

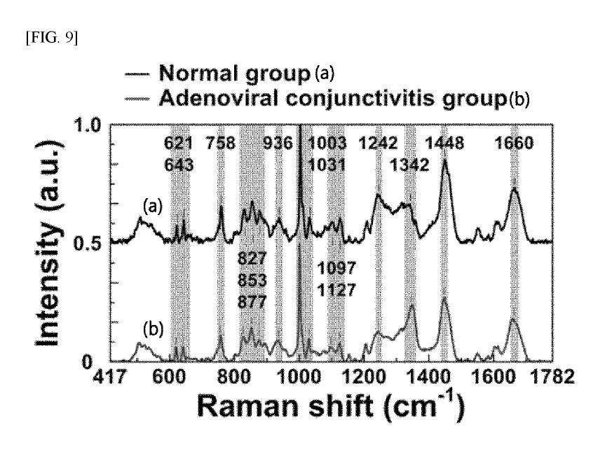

As shown in FIGS. 7A-D, it was confirmed that the signal intensity change depending on the amount of tear was due to changes in evaporation processor capillary flow rates. FIGS. 7A-D show the results of analyzing the movement of particles from the center to the ring part with respect to an arbitrary time change using a finite element analysis technique.

When FIGS. 6A-F is more specifically compared, overall, although the intensity is shown to be high in the R zone, the difference thereof was removed when the normalization process was performed as described above. The parts indicated by arrows in the spectrum correspond to 1242 cm⁻¹ and 1342 cm⁻¹, respectively, and although the spectrum was measured from the samples taken from patients with adenoviral conjunctivitis, it was confirmed that the spectral pattern in the R zone, in particular, the relative peak intensity at the two wavelengths was different from the pattern appearing in the other zones.

The DCD-SERS spectra measured at 10 different points in the same zone of the same sample were superimposed and shown in FIGS. 8A-B. The mean pairwise linear correlation coefficient of the 10 measured DCD-SERS spectra derived using the CORR function of MATLAB software was 99.29±0.04%. In particular, the intensity variations of the DCD-SERS spectrum at 1242 cm⁻¹ and 1342 cm⁻¹, which are regions of interest, were 340±26.47 and 275.88±20.2, respectively, and the coefficients of variation (CV, RSD) were 7.77% 7.37%, respectively. These results show that the method for preparing samples as suggested in the present invention provides a very consistent DCD-SERS spectrum. The Raman spectra measured after 14 weeks for the samples prepared by the suggested method showed no significant difference in the peak shift or intensity.

FIG. 9 shows the DCD-SERS spectra of the samples collected from non-infected persons and adenoviral conjunctivitis patients. The obtained spectra were compared and the results were summarized for each of the Raman peaks, and the characteristics of each peak were assigned. Specifically, it was confirmed that the peak at 621 cm⁻¹ was related to five-membered ring deformation, the peak at 643 cm⁻¹ was related to thymine ring angle bending, the peak at 758 cm⁻¹ was related to tryptophan ring breath, the peak at 853 cm⁻¹ was related to tyrosine ring breath, the peak at 877 cm⁻¹ was related to symmetric C—C stretching in lipids, the peak at 936 cm⁻¹ was related to the C—C skeleton in proteins, the peak at 1003 cm⁻¹ was related to phenylalanine symmetric ring breath, the peak at 1031 cm⁻¹ was related to phenylalanine, the peak at 1097 cm⁻¹ was related to O—P—O stretching, the peak at 1127 cm⁻¹ was related to C—N and C—C stretching of proteins, the peak at 1242 cm⁻¹ was related to the amide III β-sheet, the peak at 1275 cm⁻¹ was related to the amide III α-helix, the peak at 1342 cm⁻¹ was related to C—H deformation in proteins, the peak at 1448 cm⁻¹ was related to C—H deformation in DNA/RNA, proteins, lipids, and carbohydrates, and the peak at 1660 cm⁻¹ was related to the amide I α-helix.

The performance of the AC biomarker in a logarithmic form on tear samples collected from non-infected persons and adenoviral conjunctivitis patients is shown in Table 2, and clinical trial results (n=100, respectively) are shown in Table 3.

TABLE 2

| | Normal group | | | | Adenoviral conjunctivitis group | | | |
|---|---|---|---|---|---|---|---|---|
| Measure | C zone | M zone | T zone | R zone | C zone | M zone | T zone | R zone |
| Sensitivity (%) | 100 | 100 | 100 | 100 | | | | |
| Specificity (%) | | | | | 100 | 100 | 100 | 100 |
| Accuracy (%) | 100 | 96 | 95 | 94 | 100 | 98 | 79 | 60 |
| Error rate (%) | 0 | 4 | 5 | 6 | 0 | 2 | 21 | 40 |
| Prevalence (%) | 100 | 96 | 95 | 94 | 0 | 2 | 21 | 40 |

TABLE 3

| Dried | Normal group | | | | | Adenoviral conjunctivitis group | | | | |
|---|---|---|---|---|---|---|---|---|---|---|
| teardrop | | TP | TN | FP | FN | Total | TP | TN | FP | FN |
| C zone | 100 | 100 | 0 | 0 | 0 | 100 | 0 | 100 | 0 | 0 |
| M zone | 100 | 96 | 0 | 4 | 0 | 100 | 0 | 98 | 0 | 2 |
| T zone | 100 | 95 | 0 | 5 | 0 | 100 | 0 | 78 | 0 | 21 |
| R | 100 | 94 | 0 | 6 | 0 | 100 | 0 | 60 | 0 | 40 |

200 DCD-SERS spectra measured from the proposed 4 zones were evaluated. First of all, the AC biomarker in tears of non-infected persons exhibited 100% sensitivity and 97% accuracy, regardless of the zone. In the non-infected group, a false positive spectrum was not observed in the C zone, but there were some false positive spectra in the other zones. Meanwhile, adenoviral conjunctivitis patients showed 100% specificity in all zones without false positives, and in the C zone and R zone, accuracies of 100% and 60% were observed, respectively. The error rate in the T zone was half the error rate for the R zone. The AC biomarkers showed a high accuracy of 99% in the C and M zones, and approximately 70% accuracy in the T and R zones.

Further, the AC biomarker depending on the severity of adenoviral conjunctivitis was evaluated. The AC biomarker performance is shown in Table 4 in a logarithmic form according to the severity of adenoviral conjunctivitis, and the clinical test results, which were separated according to the severity, are shown in Table 5. Specifically, whereas the accuracy for mild adenoviral conjunctivitis in the R zone was 27%, in the case of severe adenoviral conjunctivitis, it was 86%, and in the other zones, the accuracy was more than approximately 80%, and in particular, in the C zone, the accuracy was 100%. These results indicate that the zone excluding the outermost ring zone (R zone) is a good Raman spectrum screening region for diagnosing adenoviral infection, and the AC marker in these zones is an excellent parameter for early diagnosis.

TABLE 4

| | Mild adenoviral conjunctivitis group | | | | Severe adenoviral conjunctivitis group | | | |
|---|---|---|---|---|---|---|---|---|
| Measure | C zone | M zone | T zone | R zone | C zone | M zone | T zone | R zone |
| Specificity (%) | 100 | 100 | 100 | 100 | 100 | 100 | 100 | 100 |
| Accuracy (%) | 100 | 96 | 80 | 27 | 100 | 100 | 78 | 86 |
| Error rate (%) | 0 | 4 | 20 | 73 | 0 | 0 | 22 | 14 |

TABLE 5

| Dried | Mild adenoviral conjunctivitis group | | | | | Severe adenoviral conjunctivitis group | | | | |
|---|---|---|---|---|---|---|---|---|---|---|
| teardrop | Total | TP | TN | FP | FN | Total | TP | TN | FP | FN |
| C zone | 50 | 0 | 50 | 0 | 0 | 50 | 0 | 50 | 0 | 0 |
| M zone | 50 | 0 | 48 | 0 | 2 | 50 | 0 | 50 | 0 | 0 |
| T zone | 50 | 0 | 40 | 0 | 10 | 50 | 0 | 39 | 0 | 11 |
| R zone | 50 | 0 | 12 | 0 | 33 | 50 | 0 | 43 | 0 | 7 |

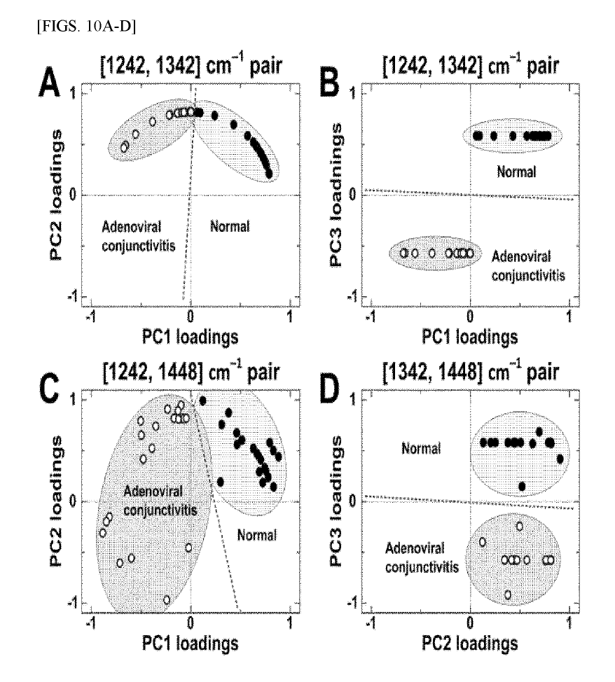

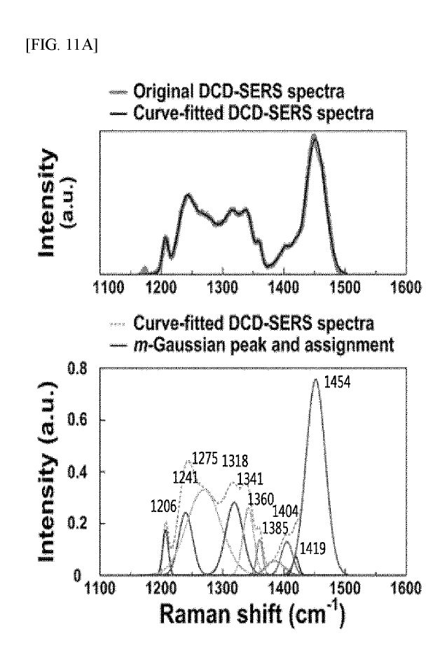

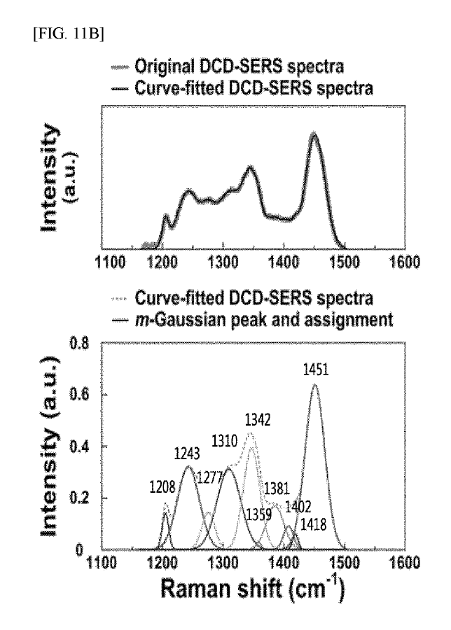

Furthermore, three PC loading profiles (PC1, PC2, and PC3) were extracted from information by the tear of the non-infected persons, the tear from the patients with adenoviral conjunctivitis, and the differences of two. This was performed in three DCD-SERS spectral vectors [1242 cm⁻¹, 1342 cm⁻¹], [1242 cm⁻¹, 1448 cm⁻¹], and [1342 cm⁻¹, 1448 cm⁻¹] in the four zones, and the results are shown in FIGS. 10A-D.

FIGS. 10A-D show loading plots of the three PC profiles in the non-infected group and the adenoviral conjunctivitis group in the C zone. As shown in FIG. 10A, in the spectrum vector [1242 cm⁻¹, 1342 cm⁻¹], there was little variation in PCI versus PC2 and PCI versus PC3, and in [1342 cm⁻¹, 1448 cm⁻¹], there was also little variation in PCI versus PC3, but in [1242 cm⁻¹, 1448 cm⁻¹], the adenoviral conjunctivitis loading profile of PC1 versus PC2 was widely distributed. Such distribution showed high AUC at [1242 cm⁻¹, 1342 cm⁻¹] and [1342 cm⁻¹, 1448 cm⁻¹] compared to [1242 cm⁻¹, 1448 cm⁻¹], regardless of the zone (Table 6).

TABLE 6

| PCA biomarker | C zone | M zone | T zone | R zone |
|---|---|---|---|---|
| [1242, 1342] cm⁻¹ | 0.9427 | 0.9007 | 0.8790 | 0.7577 |
| [1242, 1448] cm⁻¹ | 0.9260 | 0.8767 | 0.8423 | 0.7517 |
| [1342, 1448] cm⁻¹ | 0.9673 | 0.9707 | 0.9550 | 0.9453 |

That is, the PCA biomarker showed AUC values of 0.9453 in the C zone and 0.8182 in the R zone, and all of the PCA biomarkers exhibited a high sensitivity of 93% or more and a detection ability of 98% for the non-infected tear samples in the R zone (Table 7). The specificity of the PCA biomarker was 95% in the C zone, 91% in the M zone, 86% in the T zone, and 76% in the R zone. These results indicate that the measurement in the C zone rather than the R zone has an excellent diagnostic ability for adenoviral conjunctivitis. The loading profiles of PC1 and PC2 in the DCD-SERS spectrum accounted for 98% of the total. The passively set linear separating lines (dashed lines) in FIGS. 10A-D could distinguish the difference between non-infected and adenoviral conjunctivitis patients. Such principal component analysis-based database classification system can be useful for early diagnosis of adenoviral conjunctivitis.

TABLE 7

| PCA | Sensitivity (%) | | | | Specificity (%) | | | |
|---|---|---|---|---|---|---|---|---|
| biomarker | C zone | M zone | T zone | R zone | C zone | M zone | T zone | R zone |
| [1242, 1342] cm⁻¹ | 100.0 | 93.3 | 100.0 | 95.0 | 89.2 | 86.6 | 81.6 | 70.0 |
| [1242, 1448] cm⁻¹ | 86.6 | 91.6 | 93.3 | 100.0 | 97.7 | 86.6 | 81.6 | 65.0 |
| [1342, 1448] cm⁻¹ | 93.3 | 95.0 | 98.3 | 98.3 | 96.6 | 98.3 | 95.0 | 91.6 |

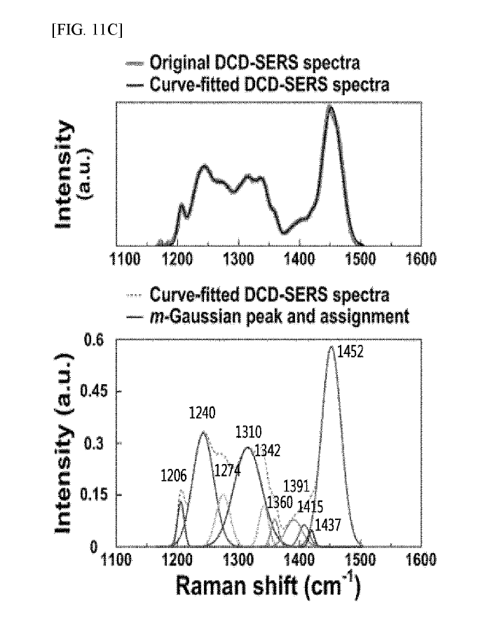

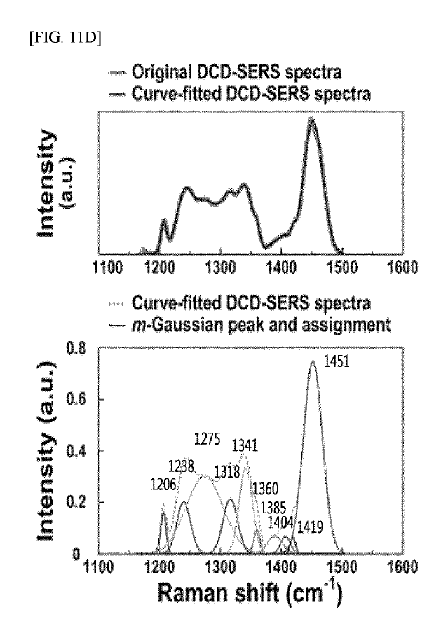

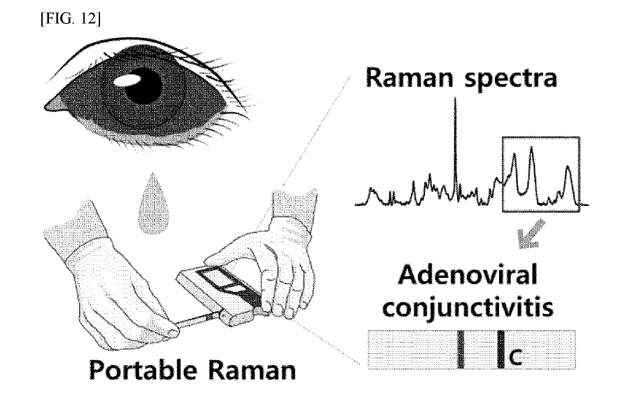

The DCD-SERS spectrum measured at wavelengths in a range of 1200 cm⁻¹ to 1500 cm⁻¹ for the C and R zones and the respective 10 Gaussian sub-peaks extracted therefrom are shown in FIGS. 11A-D. The curve-fitted DCD-SERS spectrum reconstructed from 10 Gaussian functions was almost identical to the measured spectrum itself. In the case of tears from non-infected persons (FIGS. 11A and 11C), the intensity at the wavelength of 1242 cm⁻¹ corresponding to the amide III β-sheet vibration in each region was stronger than that at the wavelength of 1342 cm⁻¹ corresponding to the C—H deformation vibration. Meanwhile, in the case of tears from the patients with adenoviral conjunctivitis, in the C zone, the intensity at the wavelength of 1342 cm⁻¹ was stronger in the opposite pattern, whereas in the R zone, differences were insignificant thereby showing a similar pattern to the normal case. Each extracted Gaussian function reflected the biochemical properties well, and four characteristic parameters of area, intensity, Raman shift, and half-width were derived from each Gaussian function (Table 8).

TABLE 8
C zone of a dried teardrop

| | Normal group | | | |
|---|---|---|---|---|
| m-Gaussian peak | Area | Intensity | Raman shift (cm⁻¹) | Half-width (cm⁻¹) |
| P1 | 2.69 ± 1.63 | 0.1655 ± 0.0457 | 1205.72 ± 0.98 | 14.69 ± 6.61 |
| P2 | 4.41 ± 1.09 | 0.1314 ± 0.0187 | 1241.37 ± 3.51 | 12.49 ± 2.45 |
| P3 | 23.30 ± 8.22 | 0.2487 ± 0.1647 | 1274.79 ± 4.34 | 72.01 ± 14.19 |
| P4 | 18.66 ± 2.91 | 0.4221 ± 0.0526 | 1315.81 ± 4.31 | 42.32 ± 11.04 |
| P5 | 7.24 ± 5.65 | 0.2709 ± 0.1395 | 1340.59 ± 1.48 | 23.01 ± 6.44 |
| P6 | 6.66 ± 5.89 | 0.2682 ± 0.2197 | 1358.15 ± 2.41 | 21.37 ± 7.34 |
| P7 | 6.05 ± 4.02 | 0.1798 ± 0.0906 | 1390.72 ± 8.56 | 28.56 ± 9.23 |
| P8 | 4.83 ± 5.59 | 0.1574 ± 0.1192 | 1413.25 ± 7.93 | 22.93 ± 11.87 |
| P9 | 2.60 ± 3.24 | 0.1516 ± 0.1480 | 1426.91 ± 13.89 | 12.77 ± 5.49 |
| P10 | 27.13 ± 1.61 | 0.7355 ± 0.1020 | 1453.86 ± 5.01 | 34.99 ± 3.96 |
| | Adenoviral conjunctivitis group | | | |
| m-Gaussian peak | Area | Intensity | Raman shift (cm⁻¹) | Half-width (cm⁻¹) |
| P1 | 3.87 ± 2.66 | 0.1550 ± 0.0522 | 1206.77 ± 1.52 | 21.31 ± 8.92 |
| P2 | 13.78 ± 2.57 | 0.3251 ± 0.0704 | 1242.77 ± 1.73 | 40.63 ± 8.61 |
| P3 | 6.50 ± 3.63 | 0.2063 ± 0.1001 | 1276.80 ± 1.31 | 28.37 ± 4.27 |
| P4 | 12.32 ± 2.41 | 0.3295 ± 0.0925 | 1310.49 ± 1.06 | 36.02 ± 6.09 |
| P5 | 11.70 ± 1.71 | 0.3831 ± 0.0697 | 1342.40 ± 3.17 | 28.97 ± 3.42 |
| P6 | 2.83 ± 2.20 | 0.1449 ± 0.1020 | 1358.97 ± 0.75 | 16.11 ± 4.39 |
| P7 | 5.62 ± 1.00 | 0.1888 ± 0.0511 | 1382.61 ± 1.79 | 28.73 ± 4.94 |
| P8 | 3.09 ± 1.32 | 0.1489 ± 0.0690 | 1403.84 ± 2.77 | 19.78 ± 2.07 |
| P9 | 2.00 ± 1.37 | 0.1154 ± 0.0644 | 1418.43 ± 1.01 | 15.15 ± 2.91 |
| P10 | 24.29 ± 4.64 | 0.6077 ± 0.1292 | 1450.99 ± 0.92 | 37.66 ± 0.84 |

M zone of a dried teardrop

| | Normal group | | | |
|---|---|---|---|---|
| m-Gaussian peak | Area | Intensity | Raman shift (cm⁻¹) | Half-width (cm⁻¹) |
| P1 | 1.59 ± 0.21 | 0.1435 ± 0.0133 | 1206.56 ± 0.06 | 10.41 ± 0.70 |
| P2 | 5.36 ± 1.39 | 0.1918 ± 0.0307 | 1239.60 ± 0.74 | 25.94 ± 2.56 |
| P3 | 21.07 ± 2.63 | 0.2831 ± 0.0090 | 1275.77 ± 0.50 | 69.85 ± 7.94 |
| P4 | 6.97 ± 1.19 | 0.2105 ± 0.0321 | 1317.84 ± 0.71 | 31.09 ± 1.55 |
| P5 | 4.61 ± 1.49 | 0.2051 ± 0.0554 | 1341.04 ± 0.50 | 20.81 ± 1.59 |
| P6 | 2.49 ± 2.12 | 0.1128 ± 0.0123 | 1355.91 ± 9.08 | 21.10 ± 18.81 |
| P7 | 1.27 ± 0.77 | 0.0559 ± 0.0137 | 1374.84 ± 11.30 | 22.85 ± 15.07 |
| P8 | 3.05 ± 0.61 | 0.1222 ± 0.0145 | 1403.58 ± 2.11 | 23.30 ± 2.15 |
| P9 | 0.77 ± 0.14 | 0.0621 ± 0.0058 | 1418.93 ± 0.93 | 11.58 ± 1.67 |
| P10 | 24.86 ± 0.46 | 0.6298 ± 0.0096 | 1451.74 ± 0.11 | 37.09 ± 0.80 |
| | Adenoviral conjunctivitis group | | | |
| m-Gaussian peak | Area | Intensity | Raman shift (cm⁻¹) | Half-width (cm⁻¹) |
| P1 | 1.98 ± 1.45 | 0.1260 ± 0.0494 | 1205.69 ± 0.79 | 14.26 ± 7.57 |
| P2 | 10.74 ± 4.54 | 0.2595 ± 0.0994 | 1241.89 ± 1.92 | 39.28 ± 13.90 |
| P3 | 5.01 ± 2.93 | 0.1555 ± 0.0762 | 1274.86 ± 3.08 | 28.73 ± 9.87 |
| P4 | 13.60 ± 6.66 | 0.2672 ± 0.1021 | 1313.51 ± 3.49 | 46.74 ± 18.54 |
| P5 | 6.69 ± 4.78 | 0.2319 ± 0.1356 | 1342.72 ± 2.91 | 24.91 ± 9.53 |
| P6 | 1.66 ± 1.65 | 0.0826 ± 0.0658 | 1358.46 ± 1.06 | 15.17 ± 8.31 |
| P7 | 3.97 ± 1.83 | 0.1262 ± 0.0529 | 1388.31 ± 5.90 | 29.26 ± 9.78 |
| P8 | 1.84 ± 0.97 | 0.0900 ± 0.0411 | 1407.25 ± 3.44 | 18.85 ± 6.24 |
| P9 | 0.82 ± 0.58 | 0.0638 ± 0.0310 | 1419.14 ± 1.169 | 11.35 ± 4.33 |
| P10 | 21.42 ± 8.14 | 0.5279 ± 0.2005 | 1451.31 ± 0.77 | 38.17 ± 11.54 |

T zone of a dried teardrop

| | Normal group | | | |
|---|---|---|---|---|
| m- Gaussian peak | Area | Intensity | Raman shift (cm⁻¹) | Half-width (cm⁻¹) |
| P1 | 1.43 ± 0.02 | 0.1205 ± 0.0004 | 1206.56 ± 0.02 | 11.17 ± 0.08 |
| P2 | 8.56 ± 0.10 | 0.2559 ± 0.0018 | 1240.34 ± 0.06 | 31.41 ± 0.16 |
| P3 | 8.84 ± 0.08 | 0.2137 ± 0.0005 | 1273.96 ± 0.05 | 38.85 ± 0.26 |
| P4 | 9.38 ± 0.17 | 0.2385 ± 0.0015 | 1315.67 ± 0.20 | 36.97 ± 0.44 |
| P5 | 4.21 ± 0.13 | 0.1830 ± 0.0024 | 1342.26 ± 0.02 | 21.62 ± 0.39 |
| P6 | 1.42 ± 0.04 | 0.0986 ± 0.0020 | 1360.06 ± 0.06 | 13.54 ± 0.08 |
| P7 | 2.56 ± 0.01 | 0.0720 ± 0.0006 | 1388.05 ± 0.10 | 33.39 ± 0.10 |
| P8 | 2.31 ± 0.00 | 0.1024 ± 0.0005 | 1406.82 ± 0.08 | 21.20 ± 0.06 |
| P9 | 0.75 ± 0.00 | 0.0617 ± 0.0000 | 1419.73 ± 0.08 | 11.42 ± 0.08 |
| P10 | 22.11 ± 0.05 | 0.5482 ± 0.0017 | 1452.16 ± 0.03 | 37.89 ± 0.03 |
| | Adenoviral conjunctivitis group | | | |
| m-Gaussian peak | Area | Intensity | Raman shift (cm⁻¹) | Half-width (cm⁻¹) |
| P1 | 1.53 ± 0.26 | 0.1338 ± 0.0108 | 1206.72 ± 0.51 | 10.67 ± 1.12 |
| P2 | 9.08 ± 3.29 | 0.2666 ± 0.0732 | 1242.24 ± 1.19 | 31.18 ± 4.16 |
| P3 | 11.17 ± 11.05 | 0.2066 ± 0.0703 | 1275.36 ± 2.44 | 44.54 ± 31.31 |
| P4 | 14.07 ± 8.36 | 0.2522 ± 0.0816 | 1314.50 ± 0.48 | 48.43 ± 21.46 |
| P5 | 6.34 ± 2.16 | 0.2417 ± 0.0579 | 1344.42 ± 3.49 | 24.15 ± 2.81 |
| P6 | 0.64 ± 0.42 | 0.0558 ± 0.0263 | 1360.42 ± 0.43 | 10.07 ± 2.15 |
| P7 | 3.08 ± 1.25 | 0.0952 ± 0.0236 | 1390.78 ± 4.46 | 29.65 ± 4.22 |
| P8 | 1.27 ± 0.38 | 0.0673 ± 0.0143 | 1408.75 ± 1.01 | 17.58 ± 1.42 |
| P9 | 0.51 ± 0.08 | 0.0514 ± 0.0058 | 1419.87 ± 0.18 | 9.35 ± 0.46 |
| P10 | 25.07 ± 1.23 | 0.6279 ± 0.0339 | 1452.46 ± 0.61 | 37.52 ± 0.24 |

R zone of a dried teardrop

| | Normal group | | | |
|---|---|---|---|---|
| m-Gaussian peak | Area | Intensity | Raman shift (cm⁻¹) | Half-width (cm⁻¹) |
| P1 | 1.61 ± 0.29 | 0.1286 ± 0.0160 | 1206.19 ± 0.40 | 11.71 ± 0.69 |
| P2 | 10.00 ± 4.76 | 0.2659 ± 0.0908 | 1240.49 ± 1.67 | 34.07 ± 4.81 |
| P3 | 7.14 ± 1.90 | 0.1833 ± 0.0256 | 1273.59 ± 1.32 | 36.37 ± 7.58 |
| P4 | 13.61 ± 4.64 | 0.2835 ± 0.0465 | 1316.58 ± 2.03 | 44.17 ± 8.83 |
| P5 | 3.76 ± 1.89 | 0.1693 ± 0.0632 | 1342.20 ± 1.13 | 20.12 ± 2.56 |
| P6 | 2.01 ± 1.41 | 0.1147 ± 0.0610 | 1358.89 ± 1.69 | 15.76 ± 3.19 |
| P7 | 2.56 ± 0.77 | 0.0762 ± 0.0182 | 1390.01 ± 4.29 | 30.98 ± 3.69 |
| P8 | 1.60 ± 0.60 | 0.0771 ± 0.0208 | 1407.82 ± 1.25 | 19.07 ± 2.41 |
| P9 | 0.58 ± 0.18 | 0.0514 ± 0.0110 | 1419.51 ± 0.43 | 10.34 ± 1.21 |
| P10 | 22.21 ± 3.72 | 0.5586 ± 0.0899 | 1451.90 ± 0.62 | 37.32 ± 0.58 |
| | Adenoviral conjunctivitis group | | | |
| m-Gaussian peak | Area | Intensity | Raman shift (cm⁻¹) | Half-width (cm⁻¹) |
| P1 | 1.80 ± 0.39 | 0.1472 ± 0.0209 | 1205.93 ± 0.62 | 11.41 ± 0.95 |
| P2 | 5.04 ± 1.92 | 0.1667 ± 0.0510 | 1237.58 ± 1.90 | 27.89 ± 2.76 |
| P3 | 16.46 ± 6.11 | 0.2353 ± 0.0363 | 1275.33 ± 0.57 | 64.44 ± 18.20 |
| P4 | 7.45 ± 2.52 | 0.2176 ± 0.0481 | 1316.50 ± 1.06 | 31.47 ± 4.35 |
| P5 | 5.92 ± 1.82 | 0.2374 ± 0.0424 | 1340.81 ± 0.89 | 22.99 ± 3.32 |
| P6 | 1.18 ± 0.31 | 0.0878 ± 0.0183 | 1359.63 ± 0.59 | 12.50 ± 1.40 |
| P7 | 2.15 ± 0.63 | 0.0677 ± 0.0141 | 1391.77 ± 6.50 | 30.00 ± 7.24 |
| P8 | 1.19 ± 0.76 | 0.0590 ± 0.0235 | 1409.21 ± 3.64 | 17.93 ± 4.92 |
| P9 | 0.55 ± 0.24 | 0.0522 ± 0.0126 | 1420.05 ± 0.84 | 9.64 ± 2.14 |
| P10 | 21.47 ± 2.23 | 0.5661 ± 0.0469 | 1451.19 ± 0.81 | 35.58 ± 0.93 |

From low wave numbers, four Gaussian functions of the second Gaussian function (the amide β-sheet at 1242 cm⁻¹), the third function (the amide III α-helix at 1275 cm⁻¹), the fifth function (C—H deformation at 1342 cm⁻¹), and the tenth function (C—H deformation at 1448 cm⁻¹) were selected as peaks for protein analysis in the tear samples.

The characteristics of the MGP biomarkers composed of selected Gaussian functions are summarized in Table 9 below. In the case of the tears from non-infected persons, the amide III β-sheet and C—H deformation were increased by 2 times by progressing from the C zone to the R zone, but the opposite pattern was observed in the tears of the adenoviral conjunctivitis patients. These changes resulted in a significant decrease (p<0.001) in the amide III α-helix of the non-infected group, and a significant increase (p<0.01) in the same of the adenoviral conjunctivitis group, but C—H deformation at 1448 cm⁻¹ did not show any significant difference in the two groups.

TABLE 9

| | Normal group (cm⁻¹) | | | | Adenoviral conjunctivitis group(cm⁻¹) | | | |
|---|---|---|---|---|---|---|---|---|
| MGP | (intensity feature)/(area feature) | | | | (intensity feature)/(area feature) | | | |
| biomarker | C zone | M zone | T zone | R zone | C zone | M zone | T zone | R zone |
| Amide III β-sheet | 1241 | 1240 | 1240 | 1240 | 1243 | 1242 | 1242 | 1238 |
| | (0.13) | (0.19) | (0.26) | (0.27) | (0.33) | (0.26) | (0.27) | (0.17) |
| | (4.40) | (5.36) | (8.56) | (10.00) | (13.78) | (10.74) | (9.08) | (5.04) |
| Amide III α-helix | 1275 | 1276 | 1274 | 1274 | 1277 | 1275 | 1275 | 1275 |
| | (0.25) | (0.28) | (0.21) | (0.18) | (0.21) | (0.16) | (0.21) | (0.24) |
| | (23.30) | (21.07) | (8.84) | (7.14) | (6.50) | (5.01) | (11.17) | (16.46) |
| C—H deformation | 1341 | 1341 | 1342 | 1342 | 1342 | 1343 | 1344 | 1341 |
| | (0.27) | (0.21) | (0.18) | (0.17) | (0.38) | (0.23) | (0.24) | (0.24) |
| | (7.24) | (4.61) | (4.21) | (3.76) | (11.70) | (6.69) | (6.34) | (5.92) |
| C—H deformation | 1454 | 1452 | 1452 | 1452 | 1451 | 1451 | 1452 | 1451 |
| | (0.74) | (0.63) | (0.55) | (0.56) | (0.61) | (0.53) | (0.63) | (0.57) |
| | (27.13) | (24.86) | (22.11) | (22.21) | (24.29) | (21.42) | (25.07) | (21.47) |

Finally, each of the Gaussian functions resolved from the multi-Gaussian model clearly showed differences in the samples collected from the non-infected persons and adenoviral conjunctivitis patients, and this indicates that MGP markers determined by the Gaussian segmentation technique can be used to qualitatively and quantitatively monitor the presence of adenoviral infection. Therefore, the method and system for detecting viral infection of the present invention can be used not only for diagnosing ophthalmic diseases caused by viral infection using tear samples, but also for diagnosing viral infection using other body fluid samples such as saliva, sweat, etc.

1. A method for providing information on the presence of viral infection, comprising:
a first step of preparing a dried tear sample on a substrate;
a second step of measuring a Raman spectrum from the dried tear sample;
a third step of extracting Gaussian sub-peaks by deconvolution of the measured Raman spectrum;
a fourth step of deriving a log value for the relative intensity ratio of a peak corresponding to an amide III β-sheet and a peak corresponding to C—H deformation by Equation 1 below; and
a fifth step of determining the sample as normal if the derived value is positive and as infected if the derived value is negative:
$$AC = {log}_{10} ( \frac{{I}_{amide III} {β}_{sheet}}{{I}_{C \text{-} Hdeform}} ) . \qquad [ Equation 1 ]$$

2. The method for providing information of claim 1, wherein the first step is performed by drop-coating deposition (DCD).

3. The method for providing information of claim 1, wherein the second step is performed by surface-enhanced Raman spectroscopy.

4. The method for providing information of claim 1, wherein the substrate is a support coated with nanoparticles.

5. The method for providing information of claim 1, wherein the measuring is performed at the central (C) zone, middle (M) zone, or secondary ring (T) zone of the dried tear sample.

6. The method for providing information of claim 1, wherein the peak corresponding to the amide III β-sheet appears in a range of 1242±10 cm⁻¹, and the peak corresponding to the C—H deformation appears in a range of 1342±10 cm⁻¹, respectively.

7. The method for providing information of claim 6, wherein the method provides information on the presence of adenoviral infection.

8. A diagnostic device for viral infection, comprising:
a detection substrate capable of providing a dried tear sample by applying a teardrop thereon;
an input unit into which the detection substrate is inserted;
a signal measuring unit for measuring a Raman signal from the inserted detection substrate;
a peak deconvolution unit for separating the measured Raman peaks into Gaussian sub-peaks;
a data processing unit for deriving a log value for a relative ratio of two peaks appearing at specific wavelengths among the separated Gaussian sub-peaks; and
a display unit for showing the derived value.

9. The diagnostic device of claim 8, wherein the signal measuring unit comprises a light source and photon detector, and optionally further comprises a mirror, lens, and a filter.
